(19) United States
(12) Patent Application Publication
(10) Pub. No.: US 2006/0129013 A1
(43) Pub. Date: Jun. 15, 2006

(54) Specific functionalization and scission of linear hydrocarbon chains

(76) Inventor: Armen N. Abazajian, Houston, TX (US)

Correspondence Address: GORDON G. WAGGETT, P.C., 3200 SOUTHWEST FREEWAY, SUITE 2355, HOUSTON, TX, 77027, US

(21) Appl. No.: 11/007,845

(22) Filed: Dec. 9, 2004

(51) Int. Cl. C07C 5/25

(52) U.S. Cl. 585/664

(57) ABSTRACT

The present invention relates generally to a method of producing single carbon number olefins and/or a narrow distribution of olefin products on demand and not as part of a distribution. The invention also relates to the olefins so produced, including, by way of example, 1-octene, and Cₙ-olefins. More specifically, in a preferred embodiment of the present invention, there is described a method for differentiating a desired internal carbon position for purposes of functionalization and scission of linear hydrocarbon chains at the desired internal carbon position. The invention provides for differentiation of the internal carbons in a linear carbon chain by introducing a methyl branch at the desired location in the linear hydrocarbon chain. The invention also provides for the production of a Cₙ-olefin from any other Cₙ-olefin. Additionally, in another preferred embodiment, there is disclosed a method of scission of the hydrocarbon chain with an internal double bond fixed in a desired tertiary location by a methyl branch to form an alpha-olefin of desired length.

### CROSS-REFERENCE TO RELATED APPLICATIONS

Not applicable.

### STATEMENT REGARDING FEDERALLY SPONSORED RESEARCH OR DEVELOPMENT

Not Applicable.

### BACKGROUND OF THE INVENTION

The present invention relates generally to a method of producing single carbon number olefins and/or a narrow distribution of olefin products on demand and not as part of a distribution. The invention also relates to the olefins so produced, including, by way of example, 1-octene, and other alpha-olefins, and the production facility. More specifically, in a preferred embodiment of the present invention, there is described a method for differentiating a desired internal carbon position for purposes of functionalization and scission of linear hydrocarbon chains at the desired internal carbon position. The invention provides for differentiation of the internal carbons in a linear carbon chain by introducing a methyl branch at the desired location in the linear hydrocarbon chain. The invention also provides for the production of a specific carbon number alpha-olefin from any other olefin, alpha-, or internal. Additionally, in another preferred embodiment, there is disclosed a method of scission of the hydrocarbon chain with an internal double bond fixed in a desired tertiary location by a methyl branch to form an alpha-olefin of desired length.

Linearity of hydrocarbon chains is a characteristic important both for biological and structural reasons. Most natural fats consist of even-numbered linear hydrocarbon chains, as if nature used ethylene as a building block. Thus linear hydrocarbon chains are usually more biodegradable than their branched, naphthenic or aromatic counterparts. In addition to biodegradability, linear chains have certain bulk physical properties, such as lubricity and reduced low-temperature viscosity which are preferred for certain applications.

While the aromatic, branched and naphthenic hydrocarbons are typically functionalized in a specific manner because of the specific reactive properties of carbons in these arrangements, linear hydrocarbon chains are difficult to functionalize on a specific internal carbon. In a linear chain, the first and the last carbons in a chain differ from the internal carbons. Also, there is a declining differentiation of second and third carbons from the end from the deeper internal carbons. The deep internal carbons (past the third carbon from the end) are energetically indistinguishable. Trewella, et at, U.S. Pat. No. 6,090,989, discusses the analytical and energetic equivalence of carbons in a linear hydrocarbon chain that are three or more carbons away from either an end or a branch. Thus, if functionalization of a specific carbon in the chain is desired, or if the hydrocarbon chain needs to be scissioned at a specific carbon, the only methods offered in the prior art are statistical methods.

For example, in the late 1970's, Shell Chemicals developed its Shell Higher Olefins Process SHOP™ process for production of alpha internal olefins from ethylene. See Shell Chemical's website: shellchemicals.com “ShellChem75th.pdf”. See also Slaugh, U.S. Pat. No. 5,243,120. As a part of this SHOP™ process, short-chain (C₄-C₈) and long-chain linear olefins (C₁₆-C₂₄) are isomerized to drive the olefin double bond to a thermodynamically determined distribution of internal positions and the olefins are metathesized together to scission the long-chain linear olefins with the short-chain olefins to a thermodynamically determined distribution of products. Because the internal carbons are thermodynamically relatively indistinguishable and because metathesis is an equilibrium reaction, beyond the third carbon the distribution of internal olefin isomers is statistic. The scissioned linear chains of the desired length are harvested from the product and the rest are recycled back to isomerization and metathesis (disproportionation) where the distribution of chain-lengths is redistributed to generate more of the harvested species.

There are a number of processes which seek to functionalize linear hydrocarbon chains on the internal carbons. One of such processes is Linear Alkyl Benzene (LAB) preparation, a well known example of which is UOP's Detal™ process, a solid catalyst, fixed-bed process in which benzene is alkylated with mono-olefins produced via UOP's Pacol™ process where normal paraffins are dehydrogenated in a vapor phase reaction to corresponding mono-olefins over a highly selective and active catalyst. See UOP's world wide website, uop.com. Another of these processes is direct oxidation of paraffins to secondary alcohols. In both of these processes, attachment of the functional group, in one case a benzyl group, in the other case a hydroxyl group, occurs randomly along the chain. Both of these products are surfactant products; therefore, optimum performance is achieved if the hydrophile is separated from the hydrophobe by a long chain. In case of the linear alkyl benzene surfactant, linear alkyl benzene is sulfonated to attach a hydrophile to the hydrocarbon molecule. Thus, the best performance with both of these methods of production is achieved if the benzyl group or the hydroxyl group is attached close to an end of the chain.

In polyethylene manufacture, linear comonomers are used to enhance the strength and to impart improved mechanical properties to polyethylene. These comonomers are one of: 1-butene, 1-hexene and 1-octene. One of the major developments in the last decade has been the advent of metallocene catalysts to polymerize lower olefins. In addition to higher turnover rates and narrower molecular distributions, metallocene catalysts exhibited an ability for much higher incorporation of comonomers into the polyethylene backbone. Higher incorporation of relatively long-chain comonomers also showed that ethylene copolymers can be elastic and take certain applications away from more expensive elastomers, especially in applications where the need for elasticity is temporary or sporadic.

Higher incorporation of comonomers into polyethylene, combined with the higher rate of growth of specialty polyethylene due to substitution of more expensive elastomers, has meant a very fast growth rate for comonomers. Of the comonomers, 1-butene is the least effective as far as imparting improved mechanical properties to the polyethylene. Thus, 1-butene use has not grown as fast as other comonomers. Therefore, the growth has concentrated on 1-octene and 1-hexene. Of these two, the use of 1-octene has grown faster than the use of 1-hexene because 1-octene is the preferred comonomer in solution polymerization, the process by which most of the specialty polyethylenes are being made.

Both 1-octene and 1-hexene are made predominantly by oligomerization of ethylene via Ziegler aluminum allyl chemistry (for example, by the following companies: Gulf/Chevron/CP Chemicals and Ethyl/Albemarle/Amoco/BP) or using a liganded Ni catalyst (Shell). In the case of 1-hexene, one other supplier has entered the market with large volumes: Sasol, which separates 1-hexene from an iron-catalyzed Fischer-Tropsch crude. Also, two of the suppliers, Shell and CP Chemicals, produce alpha-olefins in a Schultz-Flory distribution which makes more hexene than octene.

In the last round of expansions, all of the major producers mentioned above expanded to capture the market growth and, as it often happens, cumulatively overexpanded. The growth rates for most of the other large volume alpha olefins (1-butene, and 1-decene to 1-octadecene (in increments of two carbon numbers) approach Gross Domestic Product (GDP). Thus, after these expansions, the producers are being limited to the demand for the basket of alpha-olefin products created. Although Sasol also entered the market with some quantities of 1-octene from the Fischer-Tropsch source, overall the supply-demand balance has resulted in 1-octene being short in the marketplace while 1-hexene and every other carbon number alpha-olefin is long. Of the four major producers, BP, Shell and CP Chemicals could increase rates to meet 1-octene demand, but that would mean that other carbon number alpha olefins must find new markets or new applications or, conversely, the price of 1-octene must reflect distress value of all other carbon number alpha-olefins. This supply—demand imbalance was exacerbated because the U.S. market for 1-decene, which is primarily used to make polyalphaolefin synthetic lubricant basestock, was drastically reduced by the severely hydroprocessed lubricant basestocks taking a large part of U.S. market share.

A similar supply—demand problem had developed several years ago (in the 1993-1998 timeframe) with 1-decene. At that time, 1-decene was being consumed at high rates for manufacture of synthetic lubricant basestocks. Then as now, the suppliers of alpha-olefins were unable to meet the demand because demand for the rest of the alpha olefin distribution (or basket of products) did not rise in concert with 1-decene demand. At the time, in lieu of a technical solution to the problem, the market solved the problem by supplementing 1-decene supply with a blend of octene, decene and dodecene, or with dodecene alone for specialty applications, which made an inferior product, but satisfied the volume demand.

Prior to the 1-decene shortage (roughly in the 1983-1993 timeframe) a shortage existed for 1-hexene. At that time, due to the developments in the polyethylene technology, 1-hexene demand began growing and, once again, its growth was limited by the supply constraints of the alpha-olefin distribution producers. Fortuitously, Sasol arrived on the scene and provided large volumes of 1-hexene purified from iron-catalyzed Fischer Tropsch crude.

In some cases, like with the Shell SHOP™ process, the prior art attempts to address the thermodynamic and energetic equivalence of deep internal carbons in a hydrocarbon chain by essentially statistical methods of shifting the reaction equilibrium to desired products by continually removing these products. However, in such cases, the yield of the desired product is limited by the statistical distribution, which in case of the SHOP™ process is only 15-25% (Slaugh, U.S. Pat. No. 5,243,120). In other cases, such as in cases of Linear Alkyl Benzene and secondary alcohols manufacture, the prior art does not attempt to address the issue of the placement of the hydrophilic end and simply accepts an inferior product. For example, to enhance the surfactant properties of a detergent, it is preferable to have the hydrophilic end of the molecule separated by the greatest possible distance from the hydrophobic end of the molecule. Thus, in this example, when designing such surfactant, it is desirable to attach a hydrophilic functional group, such as benzene sulfonate, close to the opposite end of the carbon chain backbone containing the hydrophobic group. It is most preferable to attach such hydrophilic group to the first, second or third carbon at the end opposite the hydrophobic end. However, with present industry techniques, there is no control on such placement. For example, with the Shell SHOP™ process, metathesis is used with short and C₁₆-C₂₄ olefins, but there is no control over where the double bond remains after isomerization. There is no control over the redistribution of the olefins. If the desired product is C₁₀-C₁₄, this cut must be distilled out of the distribution, and the lights fraction and heavies fraction of the distribution are run again until another equilibrium redistribution is achieved and the process is repeated in iterative fashion.

Additionally, other representative prior art blindly skeletally isomerizes to achieve a methyl (CH₃) branch on the carbon chain for purposes of altering the bulk properties of the molecule. However, the functionalization process employed in this prior art methodology occurs in such a way as to exclude the added CH₃. For example, in the UOP methods, when functionalizing to alter bulk properties, i.e., surfactant properties, it is taught to attach the functional hydrophile away from the added methyl branch since the branch was being added for purposes of altering the bulk properties of the molecule, not as, for example, the location of the hydrophilic functional group. See, e.g., Cripe, T., et al, “Improved Alkyl Benzene Surfactants: Molecular Design and Solution Physical Chemical Properties”, The Procter and Gamble Company.

Given that most methyl branch additions end up at the first, second or third carbons of the carbon chain, what is needed is a method that advantageously uses this tertiary carbon site as the focal point for subsequent chemical reactions, rather than relying on random, or blind addition of a methyl group followed by statistical or equilibrium distribution of various products created from subsequent chemical reactions.

Thus a method is needed that would differentiate a desired internal carbon position for the purposes of functionalization and scission of linear hydrocarbon chains at that desired internal carbon position.

Also, the production of specific alpha-olefins in general and, currently, 1-octene in particular as a part of a Schultz-Flory or Poisson distribution, limits the ability of the suppliers to respond to high growth rates at only one carbon number. This condition seems to be perennial at one carbon or another for the last twenty or so years.

Thus a method is needed to make single carbon number olefins and a narrow distribution of olefin products on demand and not as a part of a distribution.

### BRIEF SUMMARY OF THE INVENTION

To address the forgoing problems, the present invention teaches a method of producing single carbon number olefins and/or a narrow distribution of olefin products on demand and not as part of a distribution. The invention also teaches a method of making olefins, for example, 1-octene, and Cₙ-olefins, using these processes. More specifically, in a preferred embodiment of the present invention, there is described a method for differentiating a desired internal carbon position for purposes of functionalization and scission of linear hydrocarbon chains at the desired internal carbon position. The invention provides for differentiation of the internal carbons in a linear carbon chain by introducing a methyl branch at the desired location in the linear hydrocarbon chain. The invention also provides for the production of a Cₙ-olefin from any other Cₙ-olefin. Additionally, in another preferred embodiment, there is disclosed a method of scission of the hydrocarbon chain with an internal double bond fixed in a desired tertiary location by a methyl branch to form an alpha-olefin of desired length.

In a preferred embodiment of the present invention, there is disclosed a process for specific functionalization of a feedstock linear hydrocarbon chain comprising the steps of introducing a methyl branch at a designed location along the linear hydrocarbon chain; and functionalizing the linear hydrocarbon chain at the designed location. The designed location of the introduced methyl branch is preferably predominantly either the second or third carbon from the end of the feedstock linear hydrocarbon chain, or in another preferred embodiment, a deep internal location on the fourth or more carbon from the end of the feedstock linear hydrocarbon chain. Another preferred designed location is the tertary carbon site on the hydrocarbon chain created by the introduced methyl branch. The feedstock linear hydrocarbon chain preferably comprises one or more mono-olefins, one or more paraffins, and/or a mixture thereof. Preferably, the feedstock paraffins are first dehydrogenated to one or more olefin products prior to the introduction of the methyl branch.

In preferred embodiments of the present invention, the feedstock linear hydrocarbon chain may comprise olefins originating from a number of sources, such as: C₄-C₃₀ alpha olefins made via ethylene oligomerization; C₃-C₃₀ even and odd carbon number alpha and internal olefins made via Fischer-Tropsch synthesis; C₃-C₃₀ even and odd carbon number predominantly internal olefins made via dehydrogenation of linear paraffins; C₃-C₃₀ even and odd carbon number predominantly internal olefins made via metathesis; and/or C₃-C₆ even and odd carbon number olefins made by thermal or stream cracking of ethane, propane or naptha. In other embodiments, the feedstock linear hydrocarbon chain comprises: C₃-C₃₀ even and odd carbon number alpha olefins; C₃-C₃₀ even and odd carbon internal olefins; and/or any mixture comprising alpha and internal olefins.

In a preferred process according to the present invention, the introduction of the methyl branch at the designed location occurs by skeletal isomerization, dimerization of olefins, or a free radical mechanism or cationic addition mechanism. The functionalization preferably occurs, in the case of paraffin feedstock, by dehydrogenation and double bond isomerization and, in the case of olefin feedstock, by double bond isomerization.

Another preferred embodiment comprises the further step of scissioning of the functionalized hydrocarbon chain at the designed location to create scission products. The scissioning may occur by oxidation, or metathesis, such as ethenolysis. A desired scission product has a carbon length Cₙ, and in one embodiment, the feedstock linear hydrocarbon chain comprises olefins in the range of Cₙ₊₂-Cₙ₊₆, where such olefins undergo skeletal isomerization and double bond isomerization prior to scissioning to result in the Cₙ product In another embodiment, the feedstock linear hydrocarbon chain comprises one or more linear alpha olefins C₃ through Cₙ₊₁, where such olefins undergo dimerization and double bond isomerization prior to scissioning to result in the Cₙ product. The scission products may be separated based on carbon length, the thus-separated scission products may be sold or recycled for use as feedstock in the process.

Another process of the present invention is the specific scissioning of a feedstock linear hydrocarbon chain comprising the steps of (a) introducing a methyl branch at a designed location along the feedstock linear hydrocarbon chain; and (b) scissioning of the hydrocarbon chain at the designed location to create scission products.

In yet another preferred embodiment, there is described a process for preparing linear alpha olefins from other linear alpha olefin and/or internal olefin feedstock by: introducing a methyl branch at a designed location along the linear hydrocarbon chain of the olefin feedstock, and scission of the linear hydrocarbon chain at the designed location to create linear alpha olefin scission products.

There is also described herein a novel process for making Cₙ alpha olefins from multiple feedstocks including linear paraffins, internal olefins, and alpha olefins alone or in combinations comprising the steps of: introducing specific methyl branching at a desired position along the linear hydrocarbon chain; and scissioning the hydrocarbon chain at the desired position to create alpha olefin scission products.

In yet another preferred embodiment, there is described a process for making products comprising specific higher alpha olefins of desired carbon numbers and isobutylene from higher alpha olefin feedstocks comprising the steps of: (a) skeletally isomerizing the higher olefin feedstocks predominantly on the second or third carbon position from the end of the chain; (b) double bond isomerizing the skeletally isomerized product of step (a); (c) scissioning the double bond isomerized product of step (b); (d) recovering the alpha olefin products; and (e) recovering the isobutylene product. In an alternative preferred embodiment, this process may comprise the additional steps of dimerizing alpha olefin products from step (d) of carbon number one higher than the desired carbon number and lower and introducing the dimers to the double bond isomerization step (b). Further, the process may include the additional steps of introducing alpha olefin products from step (d) of carbon number two or higher than the desired carbon number to the skeletal isomerization step (a). In this embodiment, the olefin feedstocks preferably comprise olefins originating from a number of sources, such as: C₈-C₂₀ alpha olefins made via ethylene oligomerization; C₈-C₂₀ even and odd carbon number alpha olefins made via Fischer-Tropsch synthesis; and/or C₃-C₃₀ even and odd carbon number alpha olefins. The scissioning step is preferably accomplished via ethylene metathesis.

In an alternate preferred embodiement, a process for making products comprising specific higher alpha olefins of desired carbon numbers and isobutylene from higher alpha olefin feedstocks advantageously comprises the steps of: (a) dimerizing the higher olefin feedstocks predominantly on the internal carbon position; (b) double bond isomerizing the dimerized product of step (a); (c) scissioning the double bond isomerized product of step (b); (d) recovering the alpha olefin products; and (e) recovering the isobutylene product. Additional steps of introducing alpha olefin products from step (d) of carbon number two higher than the desired carbon number and higher to the double bond isomerization step (b) may be employed as well as the additional steps of introducing alpha olefin products from step (d) of carbon number one higher and lower than the desired carbon number to the dimerization step (a). Preferably, the olefin feedstocks comprise olefins originating from a number of sources: C₃-C₂₀ alpha olefins made via ethylene oligomerization; C₃-C₂₀ even and odd carbon number alpha olefins made via Fischer-Tropsch synthesis; C₃-C₂₀ even and odd carbon number alpha olefins made via dehydration of primary and secondary alcohols; and/or C₃-C₆ even and odd carbon number alpha olefins made by thermal or stream cracking of ethane, propane or naptha Preferably, the olefin feedstocks comprise olefins in the range of Cₙ₊₂-Cₙ₊₆, where n is the carbon number of a desired olefin end product, and where such olefin feedstocks undergo skeletal isomerization and double bond isomerization prior to scissioning to result in a Cₙ desired product Alternatively, the olefin feedstocks may comprise one or more linear alpha olefins C₃ through Cₙ₊₁, where n is the carbon number of a desired olefin end product, and where such olefin feedstocks undergo dimerization and double bond isomerization prior to scissioning to result in a Cₙ desired product.

Where paraffin feedstock is utilized, the methodology of the present invention includes a process for making products comprising specific higher alpha olefins and isobutylene from specifically branched paraffins feedstock comprising the steps of: dehydrogenating the specifically branched paraffins; double-bond isomerizing the dehydrogenated products; scissioning the isomerized products; recovering the desired carbon number alpha olefin; and dimerizing alpha olefin products of carbon number one higher than the desired carbon number and recycling such dimers to the double bond isomerization step. This process may include the additional steps of separating the scission products based on carbon length and, if desired, recycling select of these separated scission products for use as part of the starting feedstocks.

Other preferred embodiments of the present invention include novel olefin products produced using the methodologies of the present invention as described herein. For example, one preferred invention discloses a specifically functionalized linear hydrocarbon chain for use in further chemical reactions created by the process of introducing a methyl branch at a designed location along a feedstock linear hydrocarbon chain; and functionalizing the linear hydrocarbon chain at the desired location. Additionally, another preferred product of the present invention is a specifically scissioned linear hydrocarbon chain product created by the process of introducing a methyl branch at a designed location along a linear hydrocarbon chain feedstock and scissioning of the hydrocarbon the designed location. Further exemplary products include a linear alpha olefin, manufactured from other linear alpha olefin and internal olefin feedstock, by the steps of introduction of a methyl branch at a designed location along the linear hydrocarbon chain of the feedstock and scission of the linear hydrocarbon chain the designed location to create linear alpha olefin scission products.

The present invention also discloses Cₙ alpha olefins products that are manufactured from multiple feedstocks including linear paraffins, internal olefins, and alpha olefins alone or in combinations by the steps of:
a. Introducing specific methyl branching at a desired position along the feedstock linear hydrocarbon chain.
b. Scissioning the hydrocarbon chain at the desired position to create the desired Cₙ alpha olefin scission products.
   The methyl branch is introduced predominantly onto the second or third carbon from the end of the linear hydrocarbon chain. In another embodiment, the methyl branch is introduced predominantly on a deep internal location on the fourth or more carbon from the end of the feedstock linear hydrocarbon chain. Another preferred desired carbon site is the tertiary carbon site on the hydrocarbon chain created by the introduced methyl branch.

### BRIEF DESCRIPTION OF THE SEVERAL VIEWS OF THE DRAWINGS

FIG. 1 shows a general block flow diagram of a process according to a preferred embodiment of the present invention.

FIG. 2 is a process flow diagram for a process according to a preferred embodiment of the present invention.

### DETAILED DESCRIPTION OF THE INVENTION

The primary object of a preferred embodiment of the present invention is dual. Firstly, a preferred embodiment of this invention provides a way to differentiate the internal carbons in a linear carbon chain by introducing a methyl branch at the desired location in the linear hydrocarbon chain. Thermodynamically, the tertiary carbon becomes a favored location for functionalization. As it is well known to those skilled in the art, tertiary carbon forms a more stable carbocation and a more stable radical. Such reactions as alkylation with an aliphatic or aromatic group are often carried out in the industry with the aid of cationic catalysis. Cationic oligomerization, alkylation and functionalization reaction mechanisms incorporate the formation of a carbocation and the more stable tertiary carbocation is considerably more likely to react with an electronegative reactant. Thus the tertiary site becomes the favored point of functionalization differentiating the tertiary carbon from other internal carbons in the chain. Although the physical properties of the hydrocarbon chain change with the introduction of a methyl branch along the chain, the methyl branch, due to its minimal steric effect, is the least likely to change the bulk physical properties of a product as compared to longer aliphatic branches or to heteroatomic functional groups. Cripe, T., et at, “Improved Alkyl Benzene Surfactants: Molecular Design and Solution Physical Chemical Properties”, The Procter and Gamble Company, discusses the positive influence of single methyl branching on surfactant properties, for example.

There are a number of methods known in the art for introducing methyl branching at a specific carbon in a linear hydrocarbon chain. Still other methods are being developed. One such method is the skeletal isomerization of paraffins or monoolefins over specific pore size zeolites or bifunctional catalysts which are specific pore zeolites impregnated with transition or noble metals which permit isomerization predominantly at the end position (second or third carbons from the end of the chain). See, e.g., Claude, Marion C. and Martens, Johan A., “Monomethyl-Branching of Long n-Alkanes in the Range from Decane to Tetracosane on Pt/H-ZSM-22 Bifunctional Catalyst”, Journal of Catalysis, 190 (2000) 39-48 and Li, Dadong, et at, “Skeletal Isomerization of Light FCC Naphtha”, Catalysis Today, 81 (2003) 65-73. Prior art shows that using a proper zeolite choice, the dominant position is on the second carbon. Thus in the case of a Linear Alkyl Benzene process or a secondary alcohol process, it would be desirable to attach the hydrophilic group near the end of the chain. A tertiary carbon would be the preferred point of attachment and yield a superior product because of its position near the end of the chain. The skeletal isomerization over zeolite predominantly rearranges the skeletal structure of a linear hydrocarbon to make a methyl branch at the preferred position. As evidenced in the prior art, both paraffins and olefins can be skeletally isomerized in this manner.

If olefins are skeletally isomerized over zeolites to provide a methyl branch predominantly at the second carbon, the tertiary carbon also represents the lowest energy position for the double bond. Since skeletal isomerization zeolites are moderately acidic, it is likely that the double bond would be isomerized to the lowest energy position at the methyl branch over the same zeolite catalyst that is used for skeletal isomerization, especially if the double bond was in the internal position before the reaction. To assure that the double bond is isomerized to the tertiary position, it may be necessary to contact the resulting end-chain methyl-branched olefin with a stronger isomerization catalyst such as alkali metal impregnated on alumina, a solid acid catalyst, or a homogeneous catalyst such as Ni/Al alkyl or Co/Al alkyl. The preferred product of skeletal isomerization and double-bond isomerization reactions then is an olefin with its double bond in the internal tertiary position as shown in Formula (I): 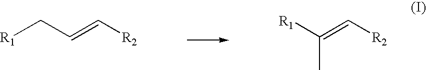
where:

R₁ above is a methyl group —CH₃ (while R₂ could be methyl, ethyl and higher alkyl groups.

In this case, the shown double bond position is the lowest energy position along the chain provided there are no other branches along the chain. In comparison, the position from the branch to R₁ would be considerably higher energy and there would be virtually no molecules with a double bond in that position in a mixture in a state of thermodynamic equilibrium. A reaction of such a tertiary olefin with a benzene or with another electron donor in the presence of a Bronsted acid or a Lewis acid cationic catalyst would functionalize the olefin at the tertiary position.

Other possible specific reactions that can occur at the tertiary site are olefin scission reactions, such as oxidation, forming aldehydes, alcohols and carboxylic acids, or metathesis reactions, where the given olefin can be disproportionated with another olefin to form an equilibrium mixture of olefins.

Skeletal isomerization of paraffins to a preferred 2-methyl position is also sufficiently described in the prior art as shown in Formula (II):

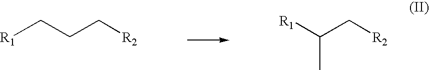

where, once again, Rₜ is a methyl or higher group and R₂ is a methyl or higher group.

Direct functionalization of the paraffinic tertiary carbon site can be undertaken with a strong Bronsted acid. Most widely used catalysts in the prior art would be peroxides for radical mechanism reactions and HF, or H₂SO₄ for carbocation reactions. The strong acid will act to protonate the tertiary carbon which will then react with an electron donor to form a bond. Oxidation to alcohols is an example of a peroxide mechanism. Alkylation to benzene alkyls or to aliphatic dimers or oligomers are catalyzed by the Bronsted acids. Alternatively, the 2-methyl-branched paraffins can be dehydrogenated to predominantly mono-olefins, for example, via UOP's Pacol®/DeFine™ combination of processes (the DeFine™ process being a liquid phase, selective hydrogenation of diolefins in the Pacol® reactor effluent to corresponding mono-olefins over a catalyst bed.) Dehydrogenation of olefins to paraffins is also otherwise widely described in the prior art. For lower carbon numbers such as C₃, C₄ and C₅, the conversions could be from 30% to 60% per pass, while for higher carbon numbers, such as C₆-C₁₅ the conversions per pass could be as low as 8%-13%. Despite the incomplete reaction, the olefins in the olefin/paraffin mixture could be isomerized and functionalized, alkylated or scissioned similarly to the olefins described above. The functionalized product would have a volatility difference with the unconverted paraffins and can be separated.

Yet another method of creating a single specific tertiary carbon site in a linear hydrocarbon chain according to the present invention is dimerization of olefins. In this method, a Lewis acid catalyst is employed, which could be a homogenenous hydrocarbon soluble catalyst, such as aluminum alkyl with or without a transition metal addition, a liquid aqueous phase catalyst like AlCl₃ or BF₃ with an oxygenated co-catalyst, a solid acid catalyst like H₃PO₄ or any of the ion exchange resins like Amberlyst™ (Rohm & Haas) or Nafion™ (DuPont). Even a weak Lewis acid such as acidic clay like Montmorillonite or an acidic zeolite can be used as a dimerization catalyst. Provided the catalyst and the reaction conditions are chosen appropriately, the product of dimerization of two olefins, especially two alpha-olefins is a single methyl branched chain. Referring to Formula (III), the initial reaction product usually is a vinylidene olefin (A.) which in the presence of an acidic catalyst quickly isomerizes to a trisubstituted position (B.). Certain catalysts and certain reaction temperatures are more likely to yield a dimethyl cross-dimer which is not desirable. The position of the double bond would depend on the acidity of the catalyst A more acidic catalyst exhibits more double bond isomerization activity and will isomerize the vinylidene to trisubstituted olefin. However, a more acidic catalyst typically is also more likely to result in more dimethyl product. Therefore, it may be necessary to separately isomerize the resulting vinylidene olefin to a trisubstituted olefin. If a single methyl branch is desired along the chain at a single predictable location, a dimer of a single olefin is required.

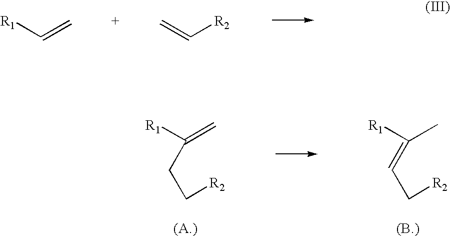

where, once again, Rₜ is a methyl or higher group and R₂ is a methyl or higher group.

The formula for a hydrogenated single olefin dimer is (n−1)-methyl-(2n−1)-ane, where n is the carbon number of a starting olefin. If the two olefins are unequal and have carbon number chains of n and m respectively, the hydrogenated dimer formula is a mixture of the following isomers:

(m−1)-methyl-(n+m−1)-ane

(m+1)-methyl-(n+m−1)-ane for n−m≧2 and

(n−1)-methyl-(n+m−1)-ane

n-methyl-(n+m−1)-ane for n−m=1

Depending on the requirements for a particular process, this mixture of two main isomers may be commercially acceptable. If not acceptable, the olefins can be segregated and dimerization can be conducted separately.

In a preferred embodiment of the present invention, when conducting the dimerization step, it is preferred to include feedstock having Cₙ₊₁ because the resulting products after scission (described herein) provide higher yields of Cₙ. Additionally, when dimerizing two olefins that are longer than butene, methyl group will be on the preferred deep internal location, three or more carbons removed from the end.

Secondly, in another preferred embodiment of the present invention, there is disclosed a method of scission of the hydrocarbon chain with an internal double bond fixed in a desired tertiary location by a methyl branch to form an alpha-olefin of desired length (for example, product (B.) in Formula (III)). Metathesis or disproportionation reactions are well known in the art. It is a reversible reaction which disproportionates alkyl groups on either side of a double bond of an olefin to an equilibrium mixture. Catalysts for the metathesis reaction are solid-supported catalysts Re or W oxides or homogeneous Ru compounds.

Referring to Formula (IV), a metathesis reaction of a longer-chain olefin with ethylene is sometimes called ethenolysis. The products of ethenolysis of a long chain olefin with ethylene are two alpha-olefin molecules with a chain length equivalent to the length of the alkyl groups on either side of the double bond plus one as depicted in products (A.) and (B.). If the internal double bond on the long chain is fixed by the methyl group which is on the second carbon of the linear hydrocarbon chain, the products of the reaction include isobutylene and an alpha-olefin of carbon number n−2, where n is the carbon number of the starting olefin. If the methyl branch is on the third or more carbon from the end of the chain, the products of the reaction are two alpha-olefins, one of which would have a methyl branch on the second carbon. Although this vinylidene olefin is highly reactive in some reactions, it is not desirable in most alpha-olefins applications. Therefore, in this case, it is necessary to isomerize the vinylidene olefin to a trisubstituted position and react it with ethylene again to form another alpha-olefin.

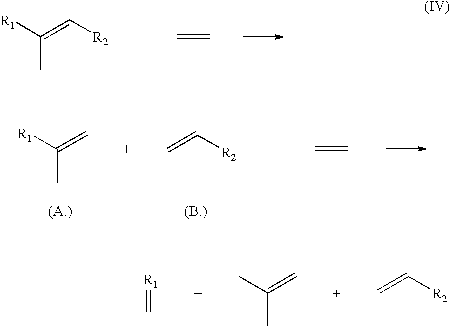

where, once again, R₁ is a methyl or higher group and R₂ is a methyl or higher group.

Assuming one starts with a m-methyl-x-n-ene, the two alpha olefins made will be 1-m-ene and 1-(n−m)-ene, where n is the carbon number of the starting olefin, m is the carbon number of the methyl branch and x is the position of the olefin bond.

If the dimer is made from a single carbon number alpha olefin, the products of consecutive ethenolysis of the dimer are isobutylene, an alpha olefin of a single carbon number lower than the feedstock monomer alpha olefin and an alpha olefin of a single carbon number higher than the feedstock monomer alpha olefin.

Because the reaction is reversible, a molar excess of ethylene must be used to retard the reverse reaction to the internal olefins.

The preferred embodiments of the invention thus disclosed can also be combined into a process to manufacture a desired alpha olefin, such as, 1-hexene, 1-octene, 1-decene or 1-dodecene as a single carbon number or as a part of a narrow distribution of products, from a range of possible feedstocks. Referring to the exemplary process to make 1-octene, as opposed to a number of processes which make 1-octene as a part of a wide distribution of co-products, the inventive process makes specifically 1-octene solely with isobutylene as a major coproduct. Isobutylene has a very large market, much larger than the market for the alpha olefin comonomers like 1-octene or lubricant feedstocks like 1-decene, as a feedstock for MTBE, isooctane, polybutylene, polyisobutylene and other specialty products. Therefore isobutylene cannot constrain 1-octene production.

Furthermore, the inventive process can use a number of feedstocks. Alpha olefins which have lower market value or for which markets are limited can be used to make another alpha olefin that is in short supply such as 1-octene now or like 1-decene several years ago. To make 1-octene, for example, 1-decene, 1-dodecene and 1-tetradecene can be skeletally isomerized specifically on the end of the chain and scissioned by ethenolysis to make 1-octene and isobutylene. While 1-decene will yield 1-octene in a single step, dodecene and tetradecene will take two and three steps respectively. 1-Hexene can be dimerized and double-bond-isomerized to a 5-methyl-(4 or 5)-undecene. Upon double scission, one of the products would be 1-heptene, which, if processed once more through dimerization and scission would yield 1-octene. 1-Butene or a mixture of 1-butene and 2-butene as it is present in Raffinate II or indeed only 2-butene as it is present in Raffinate III can be taken through four cycles of dimerization and scission and yield 1-octene. Some of the other alternative feedstocks are 1-pentene and 1-heptene available from Fischer-Tropsch manufacturers of alpha-olefins. These can be dimerized and scissioned to yield 1-octene.

Yet another possible source of feedstock is the linear paraffins, C₁₀ or C₁₀₋₁₄, which are currently being supplied commercially for dehydrogenation to olefins. These paraffins can be skeletally isomerized either as paraffins or olefins after dehydrogenation and scissioned via ethenolysis to yield 1-octene. While C₁₀ will yield 1-octene in a single step, C₁₂ and C₁₄ would take 2 and 3 steps respectively. On the other hand, skeletal isomerization and scission of C₁₁ and C₁₃ would result in odd carbon number alpha olefins which would have to be dimerized and scissioned yet again to yield 1-octene.

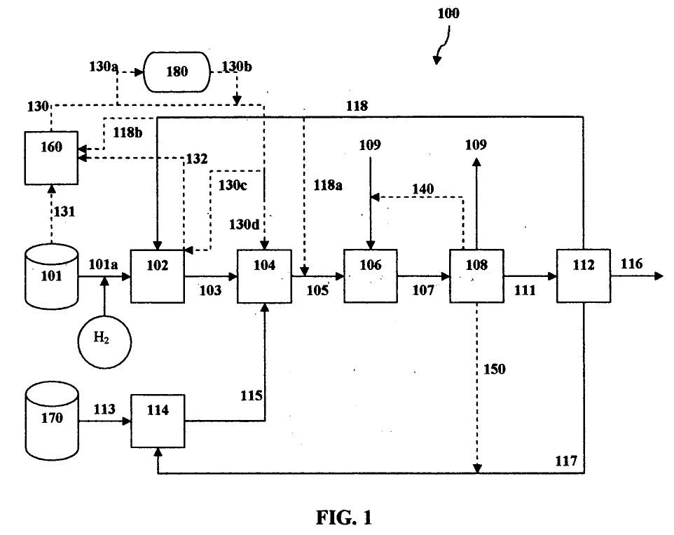

Referring now to FIG. 1, there are depicted preferred embodiments of a novel process 100 for specific functionalization and scission of linear hydrocarbon chains to produce desired end products. In this process, feedstock 101 of desired olefin(s) (carbon numbers Cₙ₊₁-Cₙ₊₆, where n is the carbon number of the desired alpha-olefin end product) are fed via suitable conduit 101a to a skeletal isomerization reactor 102 at a preferred WHSV (Weight Hourly Space Velocity) of 0.1-5 h⁻¹. Skeletal isomerization reactions and reactors are well known in the art. See, e.g., the following references for a discussion of skeletal isomerization of paraffins and olefins: Corma, A., and Orchilles, A. V., “Current Views on the Mechanism of Catalytic Cracking”, Mimporous and Mesopomus Materials, 35-36 (2000) 21-30; Harmer, Claude, Marion C. and Martens, Johan A., “Monomethyl-Branching of Long n-Alkanes in the Range from Decane to Tetracosane on Pt/H-ZSM-22 Bifunctional Catalyst”, Journal of Catalysis, 190 (2000) 39-48; Martens, Johan A., et al., “Attempts to Rationalize the Distribution of Hydrocracked Products. III. Mechanistic Aspects of Isomerization and Hydrocracking of Branched Alkanes on Ideal Bifunctional Large-Pore Zeolite Catalysts”, Catalysis Today, 1 (1987) 435-453; Li, Dadong, et al, “Skeletal Isomerization of Light FCC Naphtha”, Catalysis Today, 81 (2003) 65-73; Benitez, Viviana M., and Figoli, Nora S., “Regeneration of W Oₓ/Al₂O₃ and Mo Oₓ/Al₂O₃ Catalyst Used During Isomerization of 1-Butene”, Catalysis Communications 4 (2003) 571-577; Johnson et al, U.S. Pat. No. 6,702,937; Santilli et al., U.S. Pat. No. 5,282,958; Van Ballegoy et al, U.S. Pat. No. 6,576,120 B1, “Catalytic Dewasing Process”, Jun. 10, 2003; Degnan et al., U.S. Pat. No. 6,652,735 B2; and Wittenbrink et al, U.S. Pat. No. 5,833,839 “High Purity Paraffin Solvent Compositions and Process for their Manufacture”, Nov. 10, 1998.

The temperatures in reactor 102 are preferably in the range of 200° C. to 485° C.; and preferred pressures are in the range of 200 to 3000 psig of H₂ pressure. Hydrogen is mixed with the olefin feed 101 and introduced into the reaction (in reactor 102) at a preferred ratio of 100-2000 vol/vol of the olefin feed. A number of preferred catalysts m a y be used in the skeletal isomerization reaction. The catalysts known in the art include varied pore zeolites such as ZSM-5, silicalite, offretite, H-offretite, ZSM-12, ZSM-21, ZSM-22, H-ZSM-22, ZSM-23, ZSM-35, ZSM-38, ZSM-48, ZSM-57, SSZ-25, SSZ-32, ferrierite, L-zeolite, as well as, SAPO-11, SAPO-41, SAPO-31, MAPO-11, MAPO-31, Theta-1, Nu-10, TEA-silicate, TPZ-3, TPZ-12, VS-12, Theta-3, ISI-4, KZ-I, EU-1, EU-4, EU-13, USY, ZSM-20, ZSM-4(omega), SAPO-37, VPI-5, MeAIPO-37, AIPO-8, cloverite, CIT-1, MCM-22, SAPO-5, MeAIPO-11, and MeAlPO-5. Alumina and silica-alumina are also usable as catalyst substrates. Mixtures of these catalyst substrates are also possible. These zeolite catalysts are known in the art to contain binder material of clay, silica, alumina, boria, zirconia, titania, silica-alumina, silica-magnesia, silica-zirconia, silica-thoria, silica-beryllia, silica-titania and mixtures thereof. Preferrably, the catalyst substrates are impregnated with Group VIII metals. Potentially, Group VIB metals may be added as co-atalysts.

In a preferred embodiment of this invention, methyl branching occurs predominantly on the second carbon of the feedstock hydrocarbon chain. A minority of methyl branches occur on the third carbon of the chain. Even smaller minority of methyl branches are deep internal, occurring on the carbons which are third and deeper from the end of the chain. The product stream 103 of the skeletal isomerization reactor 102 is preferably sent next to a double-bond isomerization reactor 104 via suitable conduit. The second and third carbon from the end of a hydrocarbon chain is typically referred to as the terminal carbon, or terminal location.

Depending on the value of n, the feedstock Cₙ₊₂ to Cₙ₊₆ olefins 101 which are fed to the skeletal isomerization step 102 of the inventive process may originate from a number of sources:
C₃-C₃₀ alpha olefins made via ethylene oligomerization;
C₃-C₃₀ even and odd carbon number alpha and internal olefins made via Fischer-Tropsch synthesis;
C₃-C₃₀ even and odd carbon number predominantly internal olefins made via dehydrogenation of linear paraffins; and/or
C₄-C₃₀ even and odd carbon number predominantly internal olefins made via metathesis.
C₄-C₃₀ even and odd carbon number alpha olefins made by dehydration of alcohols.

Thus, the olefin feedstock used for embodiments of the present invention can also comprise olefins that are:
C₃-C₃₀ even and odd carbon number alpha olefins;
C₃-C₃₀ even and odd carbon internal olefins; and/or any mixture comprising alpha and internal olefins.

In yet more preferred embodiment of the present invention the olefin feedstock can comprise the following olefins originating from a number of sources:
C₄-C₁₆ alpha olefins made via ethylene oligomerization;
C₃-C₁₆ even and odd carbon number alpha and internal olefins made via Fischer-Tropsch synthesis;
C₃-C₁₆ even and odd carbon number predominantly internal olefins made via dehydrogenation of linear paraffins;
C₃-C₁₆ even and odd carbon number predominantly internal olefins made via metathesis; and/or
C₃-C₆ even and odd carbon number olefins made by thermal or stream cracking of ethane, propane or naptha.

All of these potential feedstocks may comprise varying amounts of paraffins as well as other potential impurities.

In an alternative preferred embodiment of this invention, linear or specifically branched paraffins are present in feedstock 101 and may be introduced via conduit 131 into a dehydrogenation unit 160 to dehydrogenate the paraffins to feedstock C₉-C₁₄ olefins by one of the processes well known to those skilled in the art. One of such processes is the UOP Pacol® process. The following exemplary references describe dehydrogenation processes: Vora, Bipin, et al., “Production of Detergent Olefins and Linear Alkylbenzenes”, Chemistry and Industry, 6 (1990) 187-192; Bloch et al., U.S. Pat. No. 3,910,994; Bloch et al., U.S. Pat. No. 3,681,442; Kocal et al, U.S. Pat. No. 5,276,231; and Marinangeli, et at, U.S. Pat. No. 6,187,981.

In a preferred dehydrogenation process 160, linear paraffins of carbon number from C₉ to C₁₄ are vapourized and contacted with a dehydrogenation catalyst typically comprising a Group VIII metal such as Pt or Pd, one or more of a group of metals such as As, Sb, Sn, Ir, Bi, an alkali metal such as Li, Na, K, Rb, Cs, and other components such as S, Cl, F on a alumina, silica, titania, or zirconia in a fixed-bed reactor. The flow arrangement can be a downflow or radial flow. Preferred dehydrogenation conditions are known in the art to include 400° C. to 600° C. temperature, operating pressures of about 10 psig to 100 psig, mol ratio of hydrogen to hydrocarbon feed of about 15:1 or less, and LHSV (Liquid Hourly Space Velocity) of 10 and above. Typically, the conversion of the paraffins to olefins is kept low, 8%-15%, to minimize undesirable by-products. Even so, some undesirable by-products such as substituted aromatics, products of cracking reaction, and randomly branched paraffins and olefins are made in the product stream 130. The resulting olefins may be separated from the paraffins by directing such products via conduit 130a to a molecular sieve separation process 180 such as UOP's OLEX® process and then fed via conduits 130b and 130c to the skeletal isomerization step 102 of the inventive process or may be fed directly from dehydrogenation reactor 160 via conduits 130 and 130c to the skeletal isomerization step of the inventive process as an olefin/paraffin mixture. In this embodiment, the product of such skeletal isomerization is directed to an equilibrium double bond isomerization reactor 104 described below via conduit 103.

In yet another preferred embodiment of this invention, the feedstock 101 C₉-C₁₄ paraffins are directed via conduit 101a to reactor 102 where such paraffins are specifically skeletally isomerized in reactor 102 prior to being directed, via conduit 132 to the dehydrogenation unit 160. In this embodiment, the product of such dehydrogenation is then directed to an equilibrium double bond isomerization reactor 104 described below via conduits 130, and 130d, and if necessary, first through separator 180.

In still another preferred embodiment, feedstock 101 comprises a distillation cut feedstock comprising C₆-C₁₄ terminally-branched paraffins which are separated from the products of a lubricant or fuel hydroisomerization process, for example as described in the references cited herein describing the hydroisomerization processes and as practiced by Chevron U.S.A. (see Johnson et al., U.S. Pat. No. 6,702,937 B2; Santilli et al., U.S. Pat. No. 5,282,958), and the licensees of their ISODEWAXING® process, such as Excel Paralubes, PetroCanada, Yukong and others. These streams are known to contain 2- or 3-methyl (terminally) branched linear hydrocarbons. In addition to the predominantly terminally-branched paraffins, these streams also contain some linear paraffins, and a large amount of naphthenics as well as some unsaturated species such as olefins and aromatics. Linear and terminally-branched paraffins may be separated from the other isomers by a molecular sieve separation process such as UOP's MOLEX® process. The product containing terminally-branched paraffins may be dehydrogenated as described above to make a mixture of predominantly terminally-branched olefins and paraffins. In this embodiment, the product containing a mixture of predominantly terminally-branched olefins and paraffins comprises stream 103 and is sent to double-bond isomerization reactor 104.

In yet another preferred embodiment of the present invention, a feedstock 170 of one or more linear alpha olefins C₄ through Cₙ₋₁ (where n is the carbon number of the desired olefin end product) are fed to a dimerization reactor 114 via suitable conduit 113. Dimerization of alpha olefins is well known in the art. A number of Lewis acid catalysts are active in cationic oligomerization of alpha olefins. The reaction conditions may vary depending on the type of catalyst used for oligomerization. The following references describe a number of preferred oligomerization technologies: Mark A., and Sun, Q., “Solid Acid Catalysis Using Ion-Exchange Resins”, Applied Catalysis A: General, 221 (2001) 45-62; Friedlander, R. H., et al., “Make Plasticizer Olefins Via n-Butene Dimerization”, Oil Gas Journal, date unknown; Heveling, Josef, U.K. Patent Application No. GB 2,200,302A; Maas, et al., U.S. Pat. No. 6,737,555 B1; Small et al., U.S. Pat. No. 6,291,733 B1; and Perego et al., U.S. Pat. No. 5,498,811.

In addition, other catalysts that can be used include BF₃ or AlCl₃ with oxygenated or aminated co-catalysts. Aluminum alkyls such as triethyl aluminum also can be used. Acidic molecular sieves can be used as oligomerization catalysts, for example, as described in Heveling, GB 2,200,302A. Other silicas, such as, montmorillonite can also be used. Solid phosphoric acid can also be used as an oligomerization catalyst. The oligomerization reaction is preferably conducted in a liquid phase. The preferred conditions are temperature from about 0° C. to about 200° C. Oligomerization reactions are highly exothermic and the reaction heat must be removed by direct or indirect cooling. Reaction pressure must be sufficient to avoid vapourization of the monomer. Reaction space velocity or residence time must be such as to preferably allow 80%, and more preferably 90% conversion of the monomer to dimer. If a homogeneous catalyst is used, the catalyst must be washed out of the reaction product. The unreacted monomer is also separated from the dimer and the dimer is fed forward to reactor 104.

Reactor 104 is an equilibrium double-bond isomerization reactor. In one embodiment, it represents a fixed bed reactor packed with a mild double-bond isomerization catalyst. Such isomerization catalysts are well known in the art. Such catalyst may include alumina, silica-alumina, silica, any of which are possibly impregnated with an oxide of an alkali metal. Alternative catalysts include a composite of silica and Nafion® (DuPont), Amberlyst® (Rohm & Haas), and other ion acidic resins used alone or in combinations. In another preferred embodiment, reactor 104 may be a continuous stirred tank reactor or a plug flow reactor utilizing homogeneous isomerization catalysts such as iron pentacarbonyl, or a combination of nickel or cobalt and aluminium alkyl. In this reaction the double bond is isomerized to a trisubstituted position. The preferred catalyst is a silica-alumina catalyst similar to an Engelhard Emcat®-100 catalyst. Preferred reaction conditions are temperatures 30° C. to 200° C. and pressures sufficient to keep the reactants in liquid phase. The preferred LHSV range is 0.1 to 5.0 hr⁻¹ and in general, the preferred LHSV is that sufficient to convert 90% or more of the vinylidene or internal olefins to a trisubstituted position next to a methyl branch.

The product 105 of the double-bond isomerization reactor 104 is fed via conduit to an ethenolysis reactor 106 where it is contacted with ethylene 109, preferably in excess, over a mixture of a metathesis and double-bond isomerization catalyst. Metathesis catalysts are well known to those skilled in the art. Among heterogeneous catalysts are oxides of rhenium or tungsten impregnated on alumina, silica or silica-alumina Among homogeneous metathesis catalysts, compounds of ruthenium can be used. The following references describe metathesis reactions: Schekler-Nahama, F., et al., “Influence of Lewis Acidity of Rhenium Heptoxide Supported on Alumina Catalyst on the Catalytic Performances in Olefin Metathesis”, Applied Catalysis A: General 167 (1998) 237-245; Herrmann et al., U.S. Pat. No. 6,552,139 B1; Coupard et al., U.S. Pat. No. 6,281,402 B1; Herrmann et al., U.S. Pat. No. 6,635,768 B1; Schwab et al., U.S. Pat. No. 6,646,172 B1; Schwab et al., U.S. Pat. No. 6,580,009 B2; Gartside et al., U.S. Pat. No. 6,683,019 B2; Powers, U.S. Pat. No. 6,768,038 B2; Gartside, U.S. Pat. No. 6,727,396 B2; Chen, U.S. Pat. No. 6,566,568 B1 and Schwab et al., U.S. Pat. No. 6,538,168 B1. The preferred catalyst for the present embodiment is rhenium oxide impregnated on alumina. The preferred reaction conditions are: temperature range 0° C. to 300° C., pressure 0 psig to 5000 psig, more preferably 150-3000 psig. Preferred LHSV is 0.05 hr⁻¹ to 50 hr⁻¹, more preferably from about 0.1 to 10 hr⁻¹. Metathesis is an equilibrium reaction. The products of the reaction of two longer-chain olefins are an undesired internal olefin. Therefore, preferably, a large molar excess of ethylene 109 is used to drive the reaction to high conversion of trisubstituted olefins to alpha olefins. The excess ethylene is preferably in an amount of 0.1 to 20 moles of ethylene over the stoichiometric ethylene requirement, more preferably 1 to 10 moles of ethylene over stoichiometric requirements. The product stream 107 of the ethenolysis reactor 106 is a mixture of alpha olefins, including isobutylene. One mole of isobutylene is made for each mole of tertiary carbon in the feed.

The product stream 107 of the ethenolysis reactor process 106 is passed into a separation unit(s) 108 where excess ethylene and products propylene, 1-butene and isobutylene are removed. Excess ethylene 140 is combined with fresh ethylene makeup 109 and fed into the ethenolysis reactor 106. Products propylene and isobutylene 142 are separated for distribution and sales. Product 1-butene 150 can either be separated for sales or fed into the dimerization reactor 114 for recycling.

Product stream 111 containing C₅ and higher alpha olefins is separated in separator 112 to remove the following fractions:

C₅-Cₙ₋₁ fraction 117 which preferably is recycled to the dimerization reactor 114;

Cₙ₊₁ and higher fraction 118 which preferably is recycled to the skeletal isomerization reactor 102.

Alternatively, the Cₙ₊₁, especially Cₙ₊₄ and higher, and higher fraction, especially C₁₂ and higher components, may be recycled via conduit 118a into the ethenolysis reactor 106 where the overall length of the hydrocarbon chain may be reduced.

In another preferred embodiment of the inventive process, product stream 118 may comprise significant paraffin component which is sent to the dehydrogenation unit 160 (via conduit 118b) or to the skeletal isomerization unit 102 followed by dehydrogenation 160.

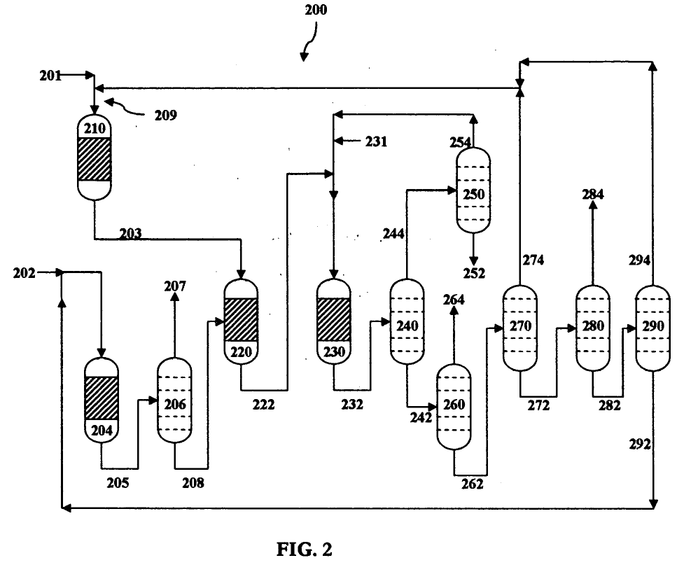

Using the teachings of the present invention, one can directly produce desired products, such as 1-octene. For example, referring now to FIG. 2, and the disclosure and techniques set out above regarding FIG. 1, there is disclosed an exemplary process flow diagram 200 for producing 1-octene according to a preferred embodiment of the present invention. A feedstock 201 of olefins is introduced into the system and proceeds to a dimerization reactor 210 as described more fully above. These reactors are typically a plug-flow, fixed catalyst-bed reactor. Preferably, the olefin feedstock 201 comprises one or more olefins in the range of C₆-C₉ (the highest carbon number preferably being one carbon number greater than the desired end product, here in this example, the C₈ olefin, 1-octene). The feedstock olefins 201 may also be supplemented with recycled olefin products from other stages of this process, thereby potentially creating a feedstock stream 209 comprising olefins from a preferred range of C₅-C₉. These feedstock and recycled olefins 201, 209 are preferably introduced into a dimerization reactor 210 where the dimerization process preferably creates olefin dimers having a single specific tertiary carbon site at a desired location in the linear hydrocarbon chain of the dimers so created. Preferably, as a result of the dimerization process, a methyl group is created in the middle of the olefin dimer carbon chain. The dimer product stream 203, including products preferably containing a single methyl branched chain, emerging from the dimerization reactor 210 is next preferably introduced into a double bond isomerization reactor 220 as described herein to isomerize the dimerized products to create an internal double bond fixed in a desired tertiary location proximate the added methyl branch. Alternatively, in another preferred embodiment, if sufficient isomerization takes place in reactor 210, 210 and 220 may physically be the same unit. The product stream 222 (containing the internal methyl branched olefins) from the double bond isomerization reactor 222 is next preferably directed to an ethenolysis reactor 230, such as that described herein, where the dimerized, isomerized olefin products are metathesized proximate the internal double bond to create two shorter chain alpha olefin end products.

The product stream 232 of the ethenolysis reactor 230 is next directed to a desired series of separation technologies to separate, e.g., by carbon chain length, the various products present, including the various C₂-C₉ alpha olefins. The separation steps can be varied depending on the end product(s) desired. For example, the ethenolysis product stream 232 can be directed into a C₂-C₃ olefin recovery/separation column 240 wherein the separated ethylene and propylene C₂-C₃ olefin product fractions 244 can then be directed to a subsequent ethylene (C₂ olefin) separation unit 250. The thus-separated ethylene (C₂ olefin) product stream 254 from separator 250 may preferably be recycled back for use as part of the ethylene feed 231 used for the ethenolysis reactor 230, while the thus-separated propylene (C₃ olefin) product stream 252 from separator 250 may be collected and distributed as product.

The non-C₂-C₃ olefin fraction 242 (e.g., C₄ and greater olefins) separated in separator 240 may next preferably proceed to a butene (C₄ olefin) separation column 260. The butene products 264, i.e., 1-butene and isobutylene, of separator 260 may be collected and distributed as product.

The non-C₄ olefin fraction 262 (e.,g., C₅ pentene and greater olefins) separated in separator 260 next preferably proceeds to a C₅-C₇ olefin separation column 270. The C₅-C₇ olefin product fractions 274 of column 270 may preferably be recycled back as feedstock olefins to combine with feedstock olefins 201 as part of an overall olefin feedstock 209 introduced into the dimerization reactor 210.

The non-C₅-C₇ olefin product fractions 272 (e.g., C₈ octene and greater olefins) of column 270 may preferably next be directed into an octene (C₈ olefin) separation unit 280, wherein the 1-octene product 284 can be collected for distribution and sale. The remaining olefin products 282 (e.g., C₉ and greater olefins) from separator 280 are preferably next directed into a C₉ separation column 290. The C₉ olefin fraction 294 separated in column 290 may preferably be recycled back as feedstock olefins to combine with feedstock olefins 201 as part of an overall olefin feedstock 209 introduced into the dimerization reactor 210.

The C₁₀ and greater olefin fraction 292, including any C₁₀ and greater paraffin products present, are preferably recycled or otherwise introduced as feedstock into a skeletal isomerization unit 204. The feedstock 202 for the skeletal isomerization unit 204 preferably includes one or more olefin products in the range of C₁₀-C₁₄ and/or one or more paraffin products in the range of C₁₀-C₁₄. The skeletal isomerization process predominantly rearranges the skeletal structure of the linear hydrocarbon feedstock olefins and/or paraffins to make a methyl branch at a preferred position. The products 205 of the skeletal isomerization unit 204 are preferably directed to a lights separation column where light-end products, such as hydrogen, methane, ethane and propane gas products 207 may be collected for use/sale. The isomerized C₁₀-C₁₄ olefin and/or paraffin products 208 separated in unit 206 may then be introduced into the double bond isomerization unit 220 for further processing as described above, the preferred product of skeletal isomerization and double-bond isomerization reactions being an olefin with its double bond in the internal tertiary position proximate the methyl branch. These isomerized products will then proceed to the ethenolysis reactor for metathesis and the further processing steps outlined above to yield the preferred alpha olefin products. Alternatively, these isomerized products can proceed to a scissioning unit for scission of the hydrocarbon chain, having an internal double bond fixed in a desired tertiary location by a methyl branch, to form an alpha-olefin of desired length.

As described above in connection with FIG. 1, with respect to the paraffin feedstock, such paraffin feedstock can undergo the steps of either skeletal isomerization followed by dehydrogenation, or visa versa, prior to the step of double bond isomerization.

The following represents an exemplary list of references.

### U.S. PATENT REFERENCES

1. Patil et at, U.S. Pat. No. 6,777,584 B2, “Selective Coupling of Terminal Olefins with Ethylene to Manufacture Linear Alpha-Olefins”, Aug. 17, 2004.

2. Powers, U.S. Pat. No. 6,768,038 B2, “Alpha Olefin Production”, Jul. 27, 2004.

3. Fenouil et al., U.S. Pat. No. 6,765,106 B2, “Process for Preparing a Branched Olefin, a Method for Using a Branched Olefin for Making a Surfactant, and a Surfactant”, Jul. 20, 2004.

4. Maas, et al., U.S. Pat. No. 6,737,555 B1, “Method for Oligomerizing C₆-Olefins”, May 18, 2004.

5. Gartside, U.S. Pat. No. 6,727,396 B2, “Process for the Production of Linear Alpha Olefins and Ethylene”, Apr. 27, 2004.

6. Johnson et al., U.S. Pat. No. 6,702,937 B2, “Process for Upgrading Fischer-Tropsch Products Using Dewaxing and Hydrofinishing”, Mar. 9, 2004.

7. Gartside et al, U.S. Pat. No. 6,683,019 B2, “Catalyst for Metathesis of Olefin”(s), Jan. 27, 2004.

8. Degnan et al., U.S. Pat. No. 6,652,735 B2, “Process for Isomerization Dewaxing of Hydrocarbon Streams”, Nov. 25, 2003.

9. Schwab et al., U.S. Pat. No. 6,646,172 B1, “Preparation of Propene”, Nov. 11, 2003.

10. Herrmann et al., U.S. Pat. No. 6,635,768 B1, “Alkylidene Complexes of Ruthenium Containing n-Heterocyclic Carbene Ligands; Use as Highly Active, Selective Catalysts for Olefin Metathesis”, Oct. 21, 2003.

11. Schwab et al., U.S. Pat. No. 6,580,009 B2, “Flexible Preparation of Propene and Hexene”, Jun. 17, 2003.

12. Van Ballegoy et al., U.S. Pat. No. 6,576,120 B1, “Catalytic Dewaxing Process”, Jun. 10, 2003.

13. Chen, U.S. Pat. No. 6,566,568 B1, “Molecular Averaging of Light and Heavy Hydrocarbons”, May 20, 2003.

14. Herrmann et al., U.S. Pat. No. 6,552,139 B1, “Process for Preparing Acyclic Olefins Using Homobimetallic and Heterobimetallic Alkylidene Complexes of Ruthenium Containing n-Heterocyclic Carbene Ligands”, Apr. 22, 2003.

15. Slaugh, U.S. Pat. No. 5,243,120, Process for the Production of Olefins, Sep. 7, 1993.

16. Schwab et al., U.S. Pat. No. 6,538,168 B1, “Preparation of C₅/C₆ Olefins”, Mar. 25, 2003.

17. Small et al., U.S. Pat. No. 6,291,733 B1, “Process for Dimerizing Olefins”, Sep. 18, 2001.

18. Coupard et al., U.S. Pat. No. 6,281,402 B1, “Alternating Process for Olefin Metathesis”, Aug. 28, 2001.

19. Marinangeli, et al., U.S. Pat. No. 6,187,981, “Process for Producing Arylalkanes and Arylalkane Sulfonates, Compositions Produced Therefrom, and Uses Thereof”, Feb. 13, 2001.

20. Trewella, et al., U.S. Pat. No. 6,090,989, “Isoparaffinic Lube Basestock Compositions”, Jul. 18, 2000.

21. Wittenbrink et al, U.S. Pat. No. 5,833,839 “High Purity Paraffin Solvent Compositions and Process for their Manufacture”, Nov. 10, 1998.

22. Perego et al., U.S. Pat. No. 5,498,811, “Process for Producing Gasolines and Jet Fuel from n-Butane”, Mar. 12, 1996.

23. Santilli et al., U.S. Pat. No. 5,282,958, “Use of Modified 5-7 A Pore Molecular Sieves for Isomerization of Hydrocarbons”, Feb. 1, 1994.

24. Kocal et al, U.S. Pat. No. 5,276,231, “Alkylaromatic process with removal of aromatic by-products”, Jan. 4, 1994.

25. Bloch et al., U.S. Pat. No. 3,910,994, “Alkylaromatic Sulfonate Detergent Process”, Oct. 7, 1994.

26. Bloch et al., U.S. Pat. No. 3,681,442, Alkylaromatic Sulfonate Detergent Process of Preparation”, Aug. 1, 1972.

### NON-U.S. PATENT REFERENCES

27. Heveling, Josef, U.K. Patent Application No. GB 2,200,302A, “Oligomerization Catalyst”, Aug. 3, 1988.

### OTHER REFERENCES

28. Cripe, T., et al, “Improved Alkyl Benzene Surfactants: Molecular Design and Solution Physical Chemical Properties”, The Procter and Gamble Company.

29. Claude, Marion C. and Martens, Johan A., “Monomethyl-Branching of Long n-Alkanes in the Range from Decane to Tetracosane on Pt/H-ZSM-22 Bifunctional Catalyst”, Journal of Catalysis, 190 (2000) 39-48.

30. Martens, Johan A., et al., “Attempts to Rationalize the Distribution of Hydrocracked Products. III. Mechanistic Aspects of Isomerization and Hydrocracking of Branched Alkanes on Ideal Bifunctional Large-Pore Zeolite Catalysts”, Cataysis Today, 1 (1987) 435-453.

31. Corma, A., and Orchilles, A. V., “Current Views on the Mechanism of Catalytic Cracking”, Microporous and Mesopomus Materials, 35-36 (2000) 21-30.

32. Schekler-Nahama, F., et al., “Influence of Lewis Acidity of Rhenium Heptoxide Supported on Alumina Catalyst on the Catalytic Performances in Olefin Metathesis”, Applied Catalysis A: General 167 (1998) 237-245.

33. Corma, A. and Garcia, H., “Organic Reactions Catalyzed Over Solid Acids”, Cataysis Today, 38 (1997) 257-308.

34. Harmer, Mark A., and Sun, Q., “Solid Acid Catalysis Using Ion-Exchange Resins”, Applied Catalysis A: General 221 (2001) 45-62.

35. Friedlander, R. H., et al., “Make Plasticizer Olefins Via n-Butene Dimerization”, Oil Gas Journal Date unknown

36. Li, Dadong, et al, “Skeletal Isomerization of Light FCC Naphtha”, Cataysis Today, 81 (2003) 65-73.

37. Benitez, Viviana M., and Figoli, Nora S., “Regeneration of W Oₓ/Al₂O₃ and Mo Oₓ/Al₂O₃ Catalyst Used During Isomerization of 1-Butene”, Catalysis Communications 4 (2003) 571-577.

38. Vora, Bipin, et al., “Production of Detergent Olefins and Linear Alkylbenzenes”, Chemistry and Industry, 6 (1990) 187-192.

39. Shell Chemical's website: shellchemicals.com “ShellChem75th.pdf”.

40. UOP's world wide website, uop.com regarding its Detal™, Pacol™, DeFine™, MOLEX® and OLEX® processes.

All references referred to herein are incorporated herein by reference. While the apparatus and methods of this invention have been described in terms of preferred embodiments, it will be apparent to those of skill in the art that variations may be applied to the process and system described herein without departing from the concept and scope of the invention. All such similar substitutes and modifications apparent to those skilled in the art are deemed to be within the scope and concept of the invention. Those skilled in the art will recognize that the method and apparatus of the present invention has many applications, and that the present invention is not limited to the representative examples disclosed herein. Moreover, the scope of the present invention covers conventionally known variations and modifications to the system components described herein, as would be known by those skilled in the art. While the apparatus, compositions and methods of this invention have been described in terms of preferred or illustrative embodiments, it will be apparent to those of skill in the art that variations may be applied to the process described herein without departing from the concept and scope of the invention. All such similar substitutes and modifications apparent to those skilled in the art are deemed to be within the scope and concept of the invention as it is set out in the following claims.

I claim:

1. A process for specific functionalization of a feedstock linear hydrocarbon chain comprising the steps of:
a. introducing a methyl branch at a designed location along the linear hydrocarbon chain; and
b. functionalizing the linear hydrocarbon chain at the designed location.

2. The process of claim 1 wherein the designed location of the introduced methyl branch is predominantly either the second or third carbon from the end of the feedstock linear hydrocarbon chain.

3. The process of claim 1 wherein the designed location is predominantly a deep internal location on the fourth or more carbon from the end of the feedstock linear hydrocarbon chain.

4. The process of claim 1 wherein the designed location is the tertiary carbon site on the hydrocarbon chain created by the introduced methyl branch.

5. The process of claim 1 wherein the feedstock linear hydrocarbon chain comprises one or more mono-olefins.

6. The process of claim 1 wherein the feedstock linear hydrocarbon chain comprises one or more paraffins.

7. The process of claim 6 wherein the feedstock paraffins are first dehydrogenated to one or more olefin products prior to the introduction of the methyl branch.

8. The process of claim 1 wherein the feedstock linear hydrocarbon chain comprises a mixture of one or more olefins with one or more paraffins.

9. The process of claim 1 wherein the feedstock linear hydrocarbon chain comprises olefins originating from a number of sources:
C₄-C₃₀ alpha olefins made via ethylene oligomerization;
C₃-C₃₀ even and odd carbon number alpha and internal olefins made via Fischer-Tropsch synthesis;
C₃-C₃₀ even and odd carbon number predominantly internal olefins made via dehydrogenation of linear paraffins;
C₃-C₃₀ even and odd carbon number predominantly internal olefins made via metathesis; and/or
C₃-C₆ even and odd carbon number olefins made by thermal or stream cracking of ethane, propane or naptha.

10. The process of claim 1 wherein the feedstock linear hydrocarbon chain comprises:
C₃-C₃₀ even and odd carbon number alpha olefins;
C₃-C₃₀ even and odd carbon internal olefins; and/or
any mixture comprising alpha and internal olefins.

11. The process of claim 1 where in step (a) the introduction of said methyl branch at said designed location occurs by skeletal isomerization.

12. The process of claim 1 where in step (b) the functionalization occurs, in the case of paraffin feedstock, by dehydrogenation and double bond isomerization.

13. The process of claim 1 where in step (b) the functionalization occurs, in the case of olefin feedstock, by double bond isomerization.

14. The process of claim 1 where in step (a) the introduction of said methyl branch at said designed location occurs by dimerization of olefins.

15. The process of claim 1 where in step (b) the functionalization of the linear hydrocarbon chain at the designed location occurs via a free radical mechanism or cationic addition mechanism.

16. The process of claim 1 comprising the further step of scissioning of the functionalized hydrocarbon chain at the designed location to create scission products.

17. The process of claim 16 wherein the scission occurs by oxidation.

18. The process of claim 16 wherein the scission occurs by metathesis.

19. The process of claim 18 wherein the methathesis is ethenolysis.

20. The process of claim 19 wherein a desired scission product has a carbon length Cₙ.

21. The process of claim 20 wherein the feedstock linear hydrocarbon chain comprises olefins in the range of Cₙ₊₂-Cₙ₊₆, where such olefins undergo skeletal isomerization and double bond isomerization prior to scissioning to result in the Cₙ product.

22. The process of claim 20 wherein the feedstock linear hydrocarbon chain comprises one or more linear alpha olefins C₃ through Cₙ₊₁, where such olefins undergo dimerization and double bond isomerization prior to -scissioning to result in the Cₙ product.

23. The process of claim 16 further comprising the step of separating the scission products based on carbon length.

24. The process of claim 16 further comprising separating the scission products based on carbon length and using select of these separated scission products as the feedstock hydrocarbon chain for step (a).

25. A process for specific scission of a feedstock linear hydrocarbon chain comprising the steps of:
a. Introducing a methyl branch at a designed location along the feedstock linear hydrocarbon chain.
b. Scissioning of the hydrocarbon chain at the designed location to create scission products.

26. The process of claim 25 wherein the designed location of the introduced methyl branch is predominantly either the second or third carbon from the end of the feedstock linear hydrocarbon chain.

27. The process of claim 25 wherein the designed location is predominantly a deep internal location on the fourth or more carbon from the end of the feedstock linear hydrocarbon chain.

28. The process of claim 25 wherein the designed location is the tertiary carbon site on the hydrocarbon chain created by the introduced methyl branch.

29. The process of claim 25 wherein the feedstock linear hydrocarbon chain comprises one or more mono-olefins.

30. The process of claim 25 wherein the feedstock linear hydrocarbon chain comprises one or more paraffins.

31. The process of claim 30 wherein the feedstock paraffins are first dehydrogenated to one or more olefin products prior to the introduction of the methyl branch.

32. The process of claim 25 wherein the feedstock linear hydrocarbon chain comprises a mixture of one or more olefins with one or more paraffins.

33. The process of claim 25 wherein the feedstock linear hydrocarbon chain comprises olefins originating from a number of sources:
C₄-C₃₀ alpha olefins made via ethylene oligomerization;
C₃-C₃₀ even and odd carbon number alpha and internal olefins made via Fischer-Tropsch synthesis;
C₃-C₃₀ even and odd carbon number predominantly internal olefins made via dehydrogenation of linear paraffins;
C₃-C₃₀ even and odd carbon number predominantly internal olefins made via metathesis; and/or
C₃-C₆ even and odd carbon number olefins made by thermal or stream cracking of ethane, propane or naptha.

34. The process of claim 25 wherein the feedstock linear hydrocarbon chain comprises:
C₃-C₃₀ even and odd carbon number alpha olefins;
C₃-C₃₀ even and odd carbon internal olefins; and/or
any mixture comprising alpha and internal olefins.

35. The process of claim 25 where in step (a) the introduction of said methyl branch at said designed location occurs by skeletal isomerization.

36. The process of claim 25 where in step (a) the introduction of said methyl branch at said designed location occurs, in the case of paraffin feedstock, by skeletal isomerization followed by dehydrogenation and double bond isomerization.

37. The process of claim 25 where in step (a) the introduction of said methyl branch at said designed location occurs, in case of an olefin feedstock, by skeletal isomerization followed by double bond isomerization.

38. The process of claim 25 where in step (a) the introduction of said methyl branch at said designed location occurs by dimerization of olefins.

39. The process of claim 25 where in step (a) the introduction of said methyl branch at said designed location occurs by dimerization followed by double bond isomerization.

40. The process of claim 25 where in step (b) the scission occurs by oxidation.

41. The process of claim 25 where in step (b) the scission occurs by metathesis.

42. The process of claim 41 wherein the methathesis is ethenolysis.

43. The process of claim 42 wherein a desired scission product has a carbon length Cₙ.

44. The process of claim 42 wherein the feedstock linear hydrocarbon chain comprises olefins in the range of Cₙ₊₂-Cₙ₊₆, where such olefins undergo skeletal isomerization and double bond isomerization prior to scissioning to result in the Cₙ product.

45. The process of claim 42 wherein the feedstock linear hydrocarbon chain comprises one or more linear alpha olefins C₃ through Cₙ₊₁, where such olefins undergo dimerization and double bond isomerization prior to scissioning to result in the Cₙ product.

46. The process of claim 25 further comprising the additional step of:
c. separating the scission products based on carbon length.

47. The process of claim 25 further comprising the additional steps of:
c. separating the scission products based on carbon length; and
d. recycling select of these separated scission products for use as feedstock linear hydrocarbon chains for step (a).

48. A process for preparing linear alpha olefins from other linear alpha olefin and/or internal olefin feedstock by:
a. Introduction of a methyl branch at a designed location along the linear hydrocarbon chain of the olefin feedstock.
b. Scission of the linear hydrocarbon chain at the designed location to create linear alpha olefin scission products.

49. The process of claim 48 wherein the designed location of the introduced methyl branch is predominantly either the second or third carbon from the end of the linear hydrocarbon chain.

50. The process of claim 48 wherein the designed location is predominantly a deep internal location on the fourth or more carbon from the end of the feedstock linear hydrocarbon chain.

51. The process of claim 48 wherein the designed location is the tertiary carbon site on the hydrocarbon chain created by the introduced methyl branch.

52. The process of claim 48 wherein the olefin feedstock comprises one or more mono-olefins.

53. The process of claim 48 wherein the olefin feedstock comprises one or more paraffins that have first been dehydrogenated to olefins.

54. The process of claim 48 wherein the olefin feedstock comprises olefins originating from a number of sources:
C₄-C₃₀ alpha olefins made via ethylene oligomerization;
C₃-C₃₀ even and odd carbon number alpha and internal olefins made via Fischer-Tropsch synthesis;
C₃-C₃₀ even and odd carbon number predominantly internal olefins made via dehydrogenation of linear paraffins;
C₃-C₃₀ even and odd carbon number predominantly internal olefins made via metathesis; and/or
C₃-C₆ even and odd carbon number olefins made by thermal or stream cracking of ethane, propane or naptha.

55. The process of claim 48 wherein the olefin feedstock comprises:
C₃-C₃₀ even and odd carbon number alpha olefins;
C₃-C₃₀ even and odd carbon internal olefins; and/or
any mixture comprising alpha and internal olefins.

56. The process of claim 48 wherein the olefin feedstock comprises a mixture of one or more olefins with one or more paraffins.

57. The process of claim 48 where in step (a) the introduction of said methyl branch at said designed location occurs by skeletal isomerization.

58. The process of claim 57 wherein the skeletal isomerization step is followed by double bond isomerization.

59. The process of claim 48 where in step (a) the introduction of said methyl branch at said designed location occurs by dimerization of olefins followed by double bond isomerization.

60. The process of claim 48 where in step (b) the scission occurs by oxidation.

61. The process of claim 48 where in step (b) the scission occurs by metathesis.

62. The process of claim 60 wherein the methathesis is ethenolysis.

63. The process of claim 48 wherein the prepared linear alpha olefin product has a carbon length Cₙ.

64. The process of claim 63 wherein the olefin feedstock comprises olefins in the range of Cₙ₊₂-Cₙ₊₆, where such olefins undergo skeletal isomerization and double bond isomerization prior to scissioning to result in the Cₙ product.

65. The process of claim 63 wherein the olefin feedstock comprises one or more linear alpha olefins C₄ through Cₙ₊₁, where such olefins undergo dimerization and double bond isomerization prior to scissioning to result in the Cₙ product.

66. The process of claim 48 further comprising the step of:
c. separating the scission products based on carbon length.

67. The process of claim 48 further comprising the steps of:
c. separating the scission products based on carbon length; and
d. recycling select of these separated scission products for use as the hydrocarbon chain for step (a).

68. A process for making Cₙ alpha olefins from multiple feedstocks including linear paraffins, internal olefins, and alpha olefins alone or in combinations comprising the steps of:
a. Introducing specific methyl branching at a desired position along the linear hydrocarbon chain.
b. Scissioning the hydrocarbon chain at the desired position to create alpha olefin scission products.

69. The process of claim 68 wherein the methyl branch is introduced onto predominantly either the second or third carbon from the end of the linear hydrocarbon chain.

70. The process of claim 68 wherein the methyl branch is introduced predominantly on a deep internal location on the fourth or more carbon from the end of the feedstock linear hydrocarbon chain.

71. The process of claim 68 wherein the desired carbon site is the tertiary carbon site on the hydrocarbon chain created by the introduced methyl branch.

72. The process of claim 68 wherein the feedstocks include one or more olefins.

73. The process of claim 68 wherein the feedstocks include one or more paraffins.

74. The process of claim 68 wherein the feedstocks include a mixture of one or more olefins with one or more paraffins.

75. The process of claim 68 wherein the introduction of said methyl branch occurs by skeletal isomerization.

76. The process of claim 68 wherein the introduction of said methyl branch occurs by dimerization of olefins.

77. The process of claim 68 where in step (b) the scission occurs by metathesis.

78. The process of claim 77 wherein the methathesis is ethenolysis.

79. The process of claim 68 wherein the olefin feedstock comprises olefins originating from a number of sources:
C₄-C₁₆ alpha olefins made via ethylene oligomerization;
C₃-C₁₆ even and odd carbon number alpha and internal olefins made via Fischer-Tropsch synthesis;
C₃-C₁₆ even and odd carbon number predominantly internal olefins made via dehydrogenation of linear paraffins;
C₃-C₁₆ even and odd carbon number predominantly internal olefins made via metathesis; and/or
C₃-C₆ even and odd carbon number olefins made by thermal or stream cracking of ethane, propane or naptha.

80. The process of claim 68 wherein the olefin feedstock comprises::
C₃-C₃₀ even and odd carbon number alpha olefins;
C₃-C₃₀ even and odd carbon internal olefins; and/or
any mixture comprising alpha and internal olefins.

81. The process of claim 68 wherein the olefin feedstock comprises olefins in the range of Cₙ₊₂-Cₙ₊₆, where such olefins undergo skeletal isomerization and double bond isomerization prior to scissioning to result in the Cₙ product.

82. The process of claim 68 wherein the olefin feedstock comprises one or more linear alpha olefins C₄ through Cₙ₊₂, where such olefins undergo dimerization and double bond isomerization prior to scissioning to result in the Cₙ product.

83. The process of claim 68 further comprising the step of:
c. separating the scission products based on carbon length.

84. The process of claim 68 further comprising the steps of:
c. separating the scission products based on carbon length; and
d. recycling select of these separated scission products for use as part of the starting feedstocks.

85. A process for making products comprising specific higher alpha olefins of desired carbon numbers and isobutylene from higher alpha olefin feedstocks comprising the steps of.
a. skeletally isomerizing the higher olefin feedstocks predominantly on the second or third carbon position from the end of the chain;
b. double bond isomerizing the skeletally isomerized product of step (a);
c. scissioning the double bond isomerized product of step (b);
d. recovering the alpha olefin products; and
e. recovering the isobutylene product.

86. The process of claim 85 comprising the additional steps of dimerizing alpha olefin products from step (d) of carbon number one higher than the desired carbon number and lower and introducing the dimers to the double bond isomerization step (b)).

87. The process of claim 85 comprising the additional steps of introducing alpha olefin products from step (d) of carbon number two or higher than the desired carbon number to the skeletal isomerization step (a).

88. The process of claim 85 wherein the olefin feedstocks comprise olefins originating from a number of sources:
C₈-C₂₀ alpha olefins made via ethylene oligomerization; and/or
C₈-C₂₀ even and odd carbon number alpha olefins made via Fischer-Tropsch synthesis.

89. The process of claim 85 wherein the olefin feedstocks:
C₃-C₃₀ even and odd carbon number alpha olefins;

90. The process of claim 85 wherein the scissioning step is accomplished via ethylene metathesis.

91. A process for making products comprising specific higher alpha olefins of desired carbon numbers and isobutylene from higher alpha olefin feedstocks comprising the steps of.
a. dimerizing the higher olefin feedstocks predominantly on the internal carbon position;
b. double bond isomerizing the dimerized product of step (a);
c. scissioning the double bond isomerized product of step (b);
d. recovering the alpha olefin products; and
e. recovering the isobutylene product.

92. The process of claim 91 comprising the additional steps of introducing alpha olefin products from step (d) of carbon number two higher than the desired carbon number and higher to the double bond isomerization step (b).

93. The process of claim 91 comprising the additional steps of introducing alpha olefin products from step (d) of carbon number one higher and lower than the desired carbon number to the dimerization step (a).

94. The process of claim 91 wherein the olefin feedstocks comprise olefins originating from a number of sources:
C₃-C₂₀ alpha olefins made via ethylene oligomerization;
C₃-C₂₀ even and odd carbon number alpha olefins made via Fischer-Tropsch synthesis;
C₃-C₂₀ even and odd carbon number alpha olefins made via dehydration of primary and secondary alcohols; and/or
C₃-C₆ even and odd carbon number alpha olefins made by thermal or stream cracking of ethane, propane or naptha.

95. The process of claim 91 wherein the olefin feedstocks comprise olefins in the range of Cₙ₊₂-Cₙ₊₆, where n is the carbon number of a desired olefin end product, and where such olefin feedstocks undergo skeletal isomerization and double bond isomerization prior to scissioning to result in a Cₙ desired product.

96. The process of claim 91 wherein the olefin feedstocks comprise one or more linear alpha olefins C₃ through Cₙ₊₁, where n is the carbon number of a desired olefin end product, and where such olefin feedstocks undergo dimerization and double bond isomerization prior to scissioning to result in a Cₙ desired product.

97. The process of claim 91 wherein the scissioning step is accomplished via ethylene metathesis.

98. A process for making products comprising specific higher alpha olefins and isobutylene from specifically branched paraffins feedstock comprising the steps of:
a. dehydrogenating the specifically branched paraffins;
b. double-bond isomerizing the dehydrogenated products of step (a);
c. scissioning the product of step (b);
d. recovering the desired carbon number alpha olefin; and
e. dimerizing alpha olefin products of carbon number one higher than the desired carbon number and recycling such dimers to step (b).

99. The process of claim 98 further comprising the additional step of:
f. separating the scission products based on carbon length.

100. The process of claim 98 further comprising the additional steps of:
f. separating the scission products based on carbon length; and
g. recycling select of these separated scission products for use as part of the starting feedstocks.

101. A specifically functionalized linear hydrocarbon chain for use in further chemical reactions created by the process of:
a. introducing a methyl branch at a designed location along a feedstock linear hydrocarbon chain; and
b. functionalizing the linear hydrocarbon chain at the desired location.

102. A specifically scissioned linear hydrocarbon chain product created by the process of:
a. Introducing a methyl branch at a designed location along a linear hydrocarbon chain feedstock.
b. Scissioning of the hydrocarbon the designed location.

103. A linear alpha olefin, manufactured from other linear alpha olefin and internal olefin feedstock, by the steps of:
a. Introduction of a methyl branch at a designed location along the linear hydrocarbon chain of the feedstock.
b. Scission of the linear hydrocarbon chain the designed location to create linear alpha olefin scission products.

104. The linear alpha olefin of claim 103 wherein the designed location of the introduced methyl branch is the second or third carbon from the end of the olefin feedstock hydrocarbon chain.

105. The linear alpha olefin of claim 103 wherein the designed location of the introduced methyl branch is a deep internal location on the fourth or more carbon from the end of the feedstock linear hydrocarbon chain.

106. The linear alpha olefin of claim 103 wherein the preferred carbon site is the tertiary carbon site on the feedstock hydrocarbon chain created by the introduced methyl branch.

107. The linear alpha olefin of claim 103 wherein the olefin feedstock comprises one or more mono-olefins.

108. The linear alpha olefin of claim 103 wherein the olefin feedstock comprises one or more paraffins that have first been dehydrogenated to olefins.

109. The linear alpha olefin of claim 103 wherein the olefin feedstock comprises a mixture of one or more olefins and one or more paraffins, such parafffins having first been dehydrogenated to olefins.

110. The process of claim 103 wherein the olefin feedstock comprises olefins originating from a number of sources:
C₄-C₃₀ alpha olefins made via ethylene oligomerization;
C₃-C₃₀ even and odd carbon number alpha and internal olefins made via Fischer-Tropsch synthesis;
C₃-C₃₀ even and odd carbon number predominantly internal olefins made via dehydrogenation of linear paraffins;
C₃-C₃₀ even and odd carbon number predominantly internal olefins made via metathesis; and/or
C₃-C₆ even and odd carbon number olefins made by thermal or stream cracking of ethane, propane or naptha.

111. The process of claim 103 wherein the olefin feedstock comprises:
C₃-C₃₀ even and odd carbon number alpha olefins;
C₃-C₃₀ even and odd carbon internal olefins; and/or
any mixture comprising alpha and internal olefins.

112. The linear alpha olefin of claim 103 where the introduction of said methyl branch at said designed location occurs by skeletal isomerization.

113. The linear alpha olefin of claim 103 where the introduction of said methyl branch at said designed location occurs by skeletal isomerization followed by double bond isomerization.

114. The linear alpha olefin of claim 103 where the introduction of said methyl branch at said designed location occurs by dimerization of olefins.

115. The linear alpha olefin of claim 103 where the introduction of said methyl branch at said designed location occurs by dimerization of olefins followed by double bond isomerization.

116. The linear alpha olefin of claim 103 where the scission occurs by metathesis.

117. The linear alpha olefin of claim 116 wherein the methathesis is ethenolysis.

118. The linear alpha olefin of claim 103 where the linear alpha olefin product has a carbon length of Cₙ.

119. The linear alpha olefin of claim 118 wherein the olefin feedstock comprises olefins in the range of Cₙ₊₂-Cₙ₊₆, where such olefins undergo skeletal isomerization and double bond isomerization prior to scissioning to result in the Cₙ product.

120. The process of claim 118 wherein the olefin feedstock comprises one or more linear alpha olefins C₄ through Cₙ₊₁, where such olefins undergo dimerization and double bond isomerization prior to scissioning to result in the Cₙ product.

121. The linear alpha olefin of claim 103 wherein the scission products are separated based on carbon length.

122. The linear alpha olefin of claim 103 wherein the scission products are separated based on carbon length; and select of these scission products are used as feedstock olefins.

123. Cₙ alpha olefins products manufactured from multiple feedstocks including linear paraffins, internal olefins, and alpha olefins alone or in combinations by the steps of:
a. Introducing specific methyl branching at a desired position along the feedstock linear hydrocarbon chain.
b. Scissioning the hydrocarbon chain at the desired position to create the desired Cₙ alpha olefin scission products.

124. The alpha olefin products of claim 123 wherein the methyl branch is introduced predominantly onto the second or third carbon from the end of the linear hydrocarbon chain.

125. The alpha olefin products of claim 123 wherein the methyl branch is introduced predominantly on a deep internal location on the fourth or more carbon from the end of the feedstock linear hydrocarbon chain.

126. The alpha olefin products of claim 123 wherein the desired carbon site is the tertiary carbon site on the hydrocarbon chain created by the introduced methyl branch.

127. The alpha olefin products of claim 123 wherein the feedstocks include one or more olefins.

128. The alpha olefin products of claim 123 wherein the feedstocks include one or more paraffins.

129. The alpha olefin products of claim 123 wherein the feedstocks include a mixture of one or more olefins with one or more paraffins.

130. The alpha olefin products of claim 123 wherein the introduction of said methyl branch occurs by skeletal isomerization.

131. The alpha olefin products of claim 123 wherein the introduction of said methyl branch occurs by dimerization of olefins.

132. The alpha olefin products of claim 123 where in step (b) the scission occurs by metathesis.

133. The alpha olefin products of claim 132 wherein the methathesis is ethenolysis.

134. The alpha olefin products of claim 123 wherein the olefin feedstock comprises olefins originating from a number of sources:
C₄-C₃₀ alpha olefins made via ethylene oligomerization;
C₃-C₃₀ even and odd carbon number alpha and internal olefins made via Fischer-Tropsch synthesis;
C₃-C₃₀ even and odd carbon number predominantly internal olefins made via dehydrogenation of linear paraffins;
C₃-C₃₀ even and odd carbon number predominantly internal olefins made via metathesis; and/or
C₃-C₆ even and odd carbon number olefins made by thermal or stream cracking of ethane, propane or naptha.

135. The alpha olefin products of claim 123 wherein the olefin feedstock comprises olefins originating from a number of sources:
C₃-C₃₀ even and odd carbon number alpha olefins;
C₃-C₃₀ even and odd carbon internal olefins; and/or
any mixture comprising alpha and internal olefins.

136. The alpha olefin products of claim 123 wherein the olefin feedstock comprises olefins in the range of Cₙ₊₂-Cₙ₊₆, where such olefins undergo skeletal isomerization and double bond isomerization prior to scissioning to result in the Cₙ product.

137. The alpha olefin products of claim 123 wherein the olefin feedstock comprises one or more linear alpha olefins C₃ through Cₙ₊₁, where such olefins undergo dimerization and double bond isomerization prior to scissioning to result in the Cₙ product.

138. The alpha olefin products of claim 123 further comprising the step of:
c. separating the scission products based on carbon length.

139. The alpha olefin products of claim 123 further comprising the steps of:
c. separating the scission products based on carbon length; and
d. recycling select of these separated scission products for use as part of the starting feedstocks.
